(19) United States
(12) Patent Application Publication
(10) Pub. No.: US 2005/0175148 A1
(43) Pub. Date: Aug. 11, 2005

(54) High Spatial Resolution X-ray and Gamma Ray Imaging System Using Crystal Diffraction Lenses

(76) Inventor: Robert K. Smither, Hinsdale, IL (US)

Correspondence Address: CHERSKOV & FLAYNIK;The Civic Opera Building, Ste 1447, 20 North Wacker Drive, Chicago, IL, 60606, US

(21) Appl. No.: 10/775,789

(22) Filed: Feb. 10, 2004

(51) Int. Cl. G21K 1/12; H05G 1/60; A61B 6/00; G01N 23/00; G21K 1/06

(52) U.S. Cl. 378/84; 378/9

(57) ABSTRACT

A method for high spatial resolution imaging of a plurality of sources of x-ray and gamma-ray radiation is provided. High quality diffracting crystals of 1 mm width are used for focussing the radiation and directing the radiation to an array of detectors which is used for analyzing their addition to collect data as to the location of the source of radiation. A computer is used for converting the data to an image. The invention also provides for the use of a multi-component high resolution detector array and for narrow source and detector apertures.

### CONTRACTUAL ORIGIN OF INVENTION

The United States Government has rights to this invention pursuant to Contract No. W-31-109-ENG-38 between the U.S. Department of Energy and the University of Chicago, representing Argonne National Laboratory.

### BACKGROUND OF THE INVENTION

1. Field of the Invention

This invention relates to a method for improving the imaging a source of radiation and to a device for imaging a source of radiation, and more specifically, this invention relates to a method and device for producing a high spatial resolution three-dimensional image of a source of x-ray and gamma-ray radiation for medical and other application by using a plurality of diffracting crystals which focus x-ray and gamma-ray radiation onto a plurality of detection devices.

2. Background of the Invention

Cancer tumor cells have high rates of metabolism and multiply rapidly. Substances injected into the body tend to migrate to locations of such high growth and become incorporated in this new growth. If the injected substance is a short-lived radioactive isotope, the location of a tumor can be detected by locating the region of high radioactivity. Aside from pinpointing tumor location, an image of the tumor is also desirable to ascertain its shape, size, and juxtaposition with adjacent structures. For many medical applications it is imperative that a tumor be detected as early as possible, and early tumors are very small in size. Thus their detection and identification requires the ability to image very small sources. Also, medical research often uses small animals, with very small organs, and the availability of devices with very high spatial resolution is of the utmost importance.

One method used to detect tumors is to first inject a body with radioactive compounds such as the Technetium isotope ^{99m}Tc, which is a 140.5 kiloelectron volt (keV) gamma emitter having a half-life of 5.9 hours. The gamma rays are detected by a large sodium iodide (NaI) scintillator crystal placed behind a collimator grid yielding at best an 8 millimeter (mm) resolution at the location of the source. The scintillator is viewed by a plurality of photomultiplier tubes and the location of a scintillation event is determined by a computer analysis of the relative intensity of the photomultiplier signals. The collimator/scintillator assembly is placed above and very close to the patient. Aside from this method yielding a low resolution of between approximately 8 mm and 1 centimeter (cm), the image produced is limited to the plane parallel to the surface of the scintillator. As such, the technique provides no depth information about the source. This deficiency can be remedied somewhat by adding another collimator/scintillator assembly below the patient, comparing the counting rate of the two scintillators, and thus estimating the position of the source along the line joining them. In the latest revision of this method the large Na I detector plus collimator is rotated around the patient, taking a plurality of images at different angles. This allows one to generate a three-dimensional image of the radiation emitting area. There are considerable additional costs associated with this method and the fact that this method has been introduced in spite of the additional costs underscores the importance of three-dimensional imaging.

Another popular imaging technique is positron emission tomography (PET), used in diagnosis and medical research. In PET, a chemical compound containing a short-lived, positron-emitting radioisotope is injected into the body. The positrons (positively charged beta particles) are emitted as the isotope decays. These particles annihilate with electrons in surrounding tissue. Each annihilation simultaneously produces two 511 (keV) gamma rays traveling in opposite directions. After passing through collimators, these two gamma rays are detected simultaneously by scintillation detectors placed at 180 degrees to each other, and on opposite sides of the patient. The signals from the detectors' photomultiplier tubes are analyzed by a computer to facilitate the production of an image of the radiation-emitting region.

Numerous drawbacks exist with scintillation detector tomography. For instance, the typical coarse resolution of no less than 8 mm often results in smaller structures being overlooked. This prevents early detection of cancerous tumors when they are least likely to have metastasized and when treatment is most likely to succeed. This is especially a disadvantage in the detection of breast cancer tumors wherein the tumors often become virulent before growing to a detectable size. Presently, mammography uses x-rays to detect tissue calcification. The assumption is made that this calcification is due to dead cancer cells and that there is a live cancer tumor in the immediate vicinity. Often however, there is no live tumor where calcification has been detected. In fact, the calcification may not have been due to a tumor at all. Unfortunately then, positive mammography results often lead to unnecessary surgical operations.

Also, because poor spacial resolution often causes images of actual small tumors to be diffuse, variations in background radiation are often mistaken for actual tumors, leading to unnecessary surgical operations. This inadvertent incorporation of background radiation is an artifact of scintillation detector use wherein the detector must be large enough to cover a given area of the body. Aside from intercepting the radiation emanating from the source under observation, however, the large detectors also detect all ambient background radiation penetrating the scintillating region and this ambient radiation is analyzed as if it had been emitted by the source under observation.

Another drawback to using imaging techniques incorporating scintillation detectors is that all of the various radiations emitted by the source are detected by the detectors. As such, a specific radiation having an energy indicative of a specific, injected isotope cannot be easily scrutinized.

Lastly, because collimators allow for the detection of only the radiation that is emitted in a very narrow direction in space, the patient has to be injected with a relatively large amount of radioactive material.

Recently, efforts have been made to improve scintillation detector tomography. Some PET instruments now achieve a resolution as small as 4 mm. Such improvements entail considerable expenditures and have the additional drawback that the improvement in resolution has come at the cost of a decrease in counting rate. This entails in turn either a longer examination time per patient or the injection of a stronger dose of radiation. Furthermore, the prospects for further improvements in resolution are limited by the fact that such improvements require collimators with ever smaller apertures, and therefore greater mass, together with lower count rates. This increase in collimator mass will increase the number of forward Compton-scattered photons in the collimators and these forward scattered photons are often indistinguishable from those emanating directly from the source.

Significant improvements in spacial resolution and in detection efficiency as well as a three dimensional location of the source using a crystal diffraction method for focusing the radiation emanating from the source was disclosed in U.S. Pat. No. 5,869,841 (1999) (granted to the same inventor as the present invention and assigned to the same assignee) and incorporated herein by reference. Because of the focussing of the radiation emitted by the source, one requires the injection of much smaller amounts of radioactive substances at a site on the patient's body in order to locate features of interest. Experiments at the inventor's laboratory have demonstrated the effectiveness of this method and have achieved a spatial resolution of 7 mm. While this is adequate under many circumstances, better spatial resolution would provide significant advantages.

Thus a need exists in the art for an improved method and device for imaging x-ray and gamma-ray sources with sufficient spacial resolution to accurately observe structures smaller than 7 mm in size, even down to 1 mm in size or less. The invented method and the resulting device must have sufficient energy resolution to allow the imaging of radiation of a selected energy to the exclusion of others. The method and device also must limit the radiation to which the patient is exposed by incorporating a redirecting or “focussing” mechanism to detect radiation emanating from a tumor while disregarding ambient levels of radiation.

### SUMMARY OF THE INVENTION

It is an object of the present invention to provide a method and a device for high spatial resolution imaging sources of gamma-ray and x-ray radiation that overcome many of the disadvantages of the prior art.

Another object of the present invention is to provide a device for improved spatial resolution in three-dimensional imaging of sources of gamma and x-ray radiation emanating from a subject. A feature of the present invention is the use of a plurality of non-coplanar assemblies each comprising one or more lenses and a detector array to record data simultaneously for analysis by a computer. An advantage of the invention is the generation of a three-dimensional image of the source while the subject is under examination.

Still another object of the present invention is to provide a method for producing a high resolution image of a radiation source located in a patient. A feature of the invention is the use of high purity and high quality diffracting crystals 1 mm wide or less. An advantage of the invention is the imaging of millimiter size sources into images of comparable size. Another advantage of the invention is the obviation of unnecessary, invasive surgical procedures to locate a tumor.

Yet another object of the present invention is to provide a radiation imaging method having a fast imaging time. A feature of the invention is the use of scintillation detectors to locate the approximate position of the radiation source and then the use of a high efficiency crystal diffraction system to produce a high resolution image of the source that can be viewed by a multi-element detector array. An advantage of the invented method is that the amount of radiation necessary to produce a high resolution image is relatively small compared to typical pure scintillation detector methods. Another advantage of the invented method is that one may not have to scan the source in order to obtain a full image thereof.

Another object of the present invention is to provide a radiation imaging method wherein there is a sharp one to one correspondence between source location and image location. A feature of the invention is the use of narrow apertures between the sources and the detectors. Another feature of the invention is the use of arrays of small size detectors to image the radiation. An advantage of the invented method is a sharp image of the radiating source as recorded by the detectors.

Another object of the present invention is to provide an economical and manageable imaging device. A feature of the invention is that each of its lenses is made of a plurality of thin, long, and arcuately bent individually mounted crystal units. An advantage of the invention is that a lens can be built rapidly and that a defective crystal in one of the lenses can be replaced quickly and at low cost. Should an individual lens become damaged in a multi-lens system, the lens can be replaced quickly and at low cost. The modular nature of the multi-lens system makes it possible to operate even though some of the crystals in a lens are damaged and even when the whole damaged lens is removed.

Still another object of the present invention is to allow the imaging system to observe radiation of a selected energy to the exclusion of other energies. A feature of the present invention is that the focal length of the lens depends very sensitively on the energy of the radiation. An advantage of the present invention is that the lens can be so constructed as to focus only radiation of the desired energy.

In brief the present invention provides a method for high spatial resolution imaging of a source of radiation comprising: a) using high purity and high quality diffracting crystals having a thickness of not more than 1 mm to focus the radiation onto a detector; b) analyzing said focused radiation to collect data as to the type and location of the radiation; and c) producing an image using the data. The invented method can be further enhanced by using a) long diffracting crystals bent in an arcuate shape, b) a multi-element detector array, and c) by positioning narrow apertures in front of the source and in front of the detector.

### BRIEF DESCRIPTION OF THE DRAWING

The present invention together with the above and other objects and advantages may best be understood from the following detailed description of the embodiment of the invention illustrated in the drawing, wherein:

FIG. 1 is an elevational view of a single coplanar array of lens/detector assemblies in accordance with features of the present invention;

FIG. 2 is a cross-sectional, plan view of two intersecting arrays of lens/detector assemblies, in accordance with features of the present invention;

FIG. 3 is a cross sectional view of a lens/detector assembly as shown in FIG. 1, taken along lines 3-3, in accordance with features of the present invention;

FIG. 4a illustrates the phenomenon commonly known as Laue Diffraction;

FIG. 4b illustrates the phenomenon commonly known as Bragg Diffraction;

FIG. 5a illustrates the effect of crystal imperfections in Laue Diffraction;

FIG. 5b illustrates the effect of crystal bending in Bragg Diffraction;

FIG. 6 is a plan view of a Laue crystal diffraction lens, in accordance with features of the present invention;

FIG. 7 is a plan view of a Bragg crystal diffraction lens, in accordance with features of the present invention;

FIG. 8a depicts a procedure for cutting a slab of predetermined orientation from a crystal of known orientation supplied by a manufacturer;

FIG. 8b depicts a procedure for cutting crystals of predetermined orientation, in accordance with features of the present invention;

FIG. 9a is a schematic views of the method for constructing a Laue diffraction lens out of a plurality of diffracting crystals, in accordance with features of the present invention;

FIG. 9b is a schematic views of the method for constructing a Laue diffraction lens out of a plurality of diffracting crystals, in accordance with features of the present invention;

FIG. 10 is a view of FIG. 6 taken along line 10-10;

FIG. 11a illustrates the effect of large diffracting crystals in a focussing lens, in accordance with features of the present invention;

FIG. 11b illustrates the effect of small diffracting crystals in a focussing lens, in accordance with features of the present invention;

FIG. 12 is a three dimensional view of a Bragg diffraction lens in accordance with features of the present invention;

FIG. 13 is a view of FIG. 12 taken along lines 12-12;

FIG. 14 is a detailed view of FIG. 12;

FIG. 15a illustrates the effect of a large aperture in front of the detector array in accordance with features of the present invention;

FIG. 15b illustrates the effect of a narrow aperture in front of the detector array in accordance with features of the present invention;

FIG. 16 illustrates the combined effect of narrow apertures in front of the detector array and in front of the source, in accordance with features of the present invention; and

FIG. 17 illustrates the effect of a many-elements detector array in accordance with features of the present invention.

### DETAILED DESCRIPTION OF THE INVENTION

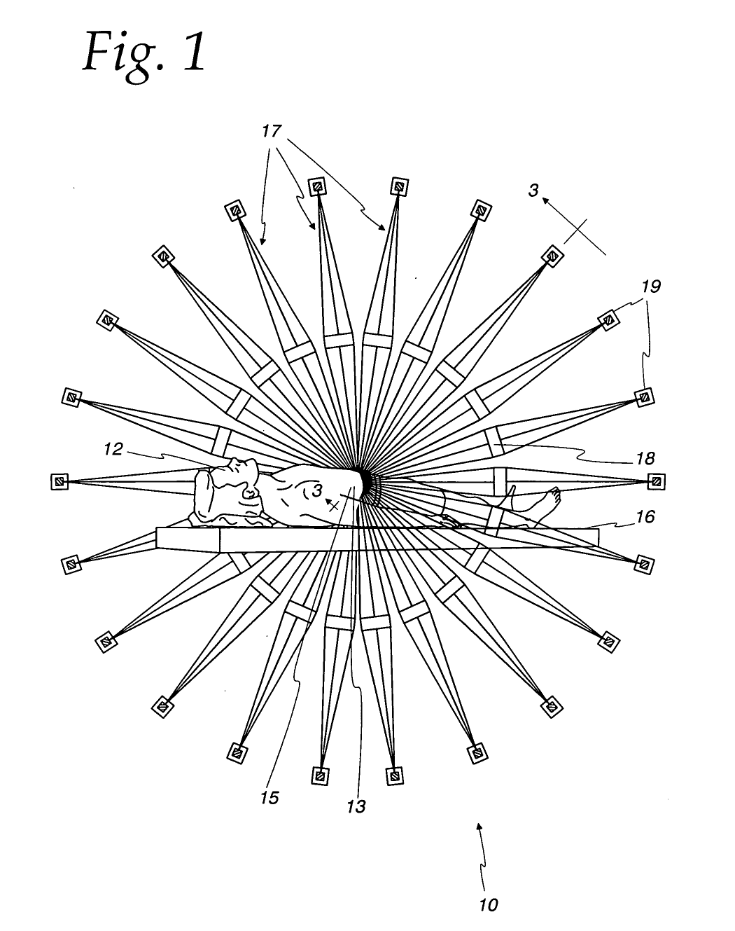

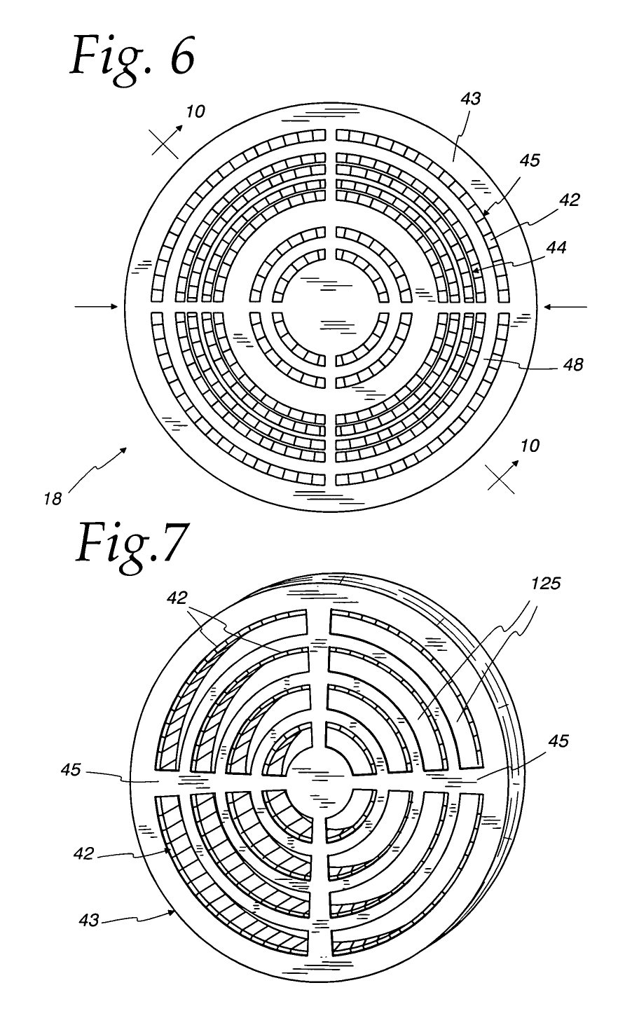

The present invention provides an improved method for imaging sources of x-ray and gamma-ray radiation. The invented method can yield a detected image 1 mm or less in size (Full Width at Half Maximum (FWHM) for a point source). The method results in a device, designated generally as numeral 10 in FIG. 1, that incorporates a plurality of lens/detector assemblies 17 to first focus and then detect radiation emanating from a radioactive source 15, such as a tumor in a patient 12 that has incorporated some radioactivity as it grows. Each lens/detector assembly 17 comprises a plurality of high efficiency and high resolution crystal diffraction lenses 18 that focus onto detector arrays 19 only the radiation of a desired energy and origin. As disclosed infra, and with reference to FIGS. 6 and 7, each lens 18 comprises a plurality of concentric rings 45, which in turn are comprised of very accurately mounted diffracting crystals. These crystals are oriented so that only radiation having a predetermined energy is focused onto a detector array 19. The detector arrays 19 of the device are shielded from unwanted radiation.

The device 10 is designed to accommodate the detection of radiation from a myriad of sources. For clarity, the radiation source 15 in the exemplary embodiment shown in FIG. 1 is a tumor that has absorbed a radio-isotope in vivo, whereby the tumor emits radiation of a predetermined wave length λ. However, other radiation sources are also appropriate, including radioisotope-impregnated fissures in a mineral or in a manufactured object, an x-ray or gamma-ray beam scattering from a target, x-rays or gamma rays produced by particle-beam bombardment of a target, and high metabolic rate regions in a living organism wherein a radioisotope has been incorporated.

After emanating from the source 15, the radiation is subjected to a means for focussing the radiation, i.e. the diffraction lens 18. The lens 18 directs the radiation to a detector array 19. The output of the detector array is analyzed by a computer. The exemplary device 10 is a plane circular array of lens/detector assemblies 17 with the source 15 situated at the center 13 of the array, the detector arrays 19 positioned along the periphery of the array, and the focussing means 18 positioned approximately medially between the source 15 and the detector assemblies 19. As noted supra, the detector arrays 19 define the periphery of the plane circular array and therefore are distally placed relative to the center 13 of the circular array and the focussing lens 18.

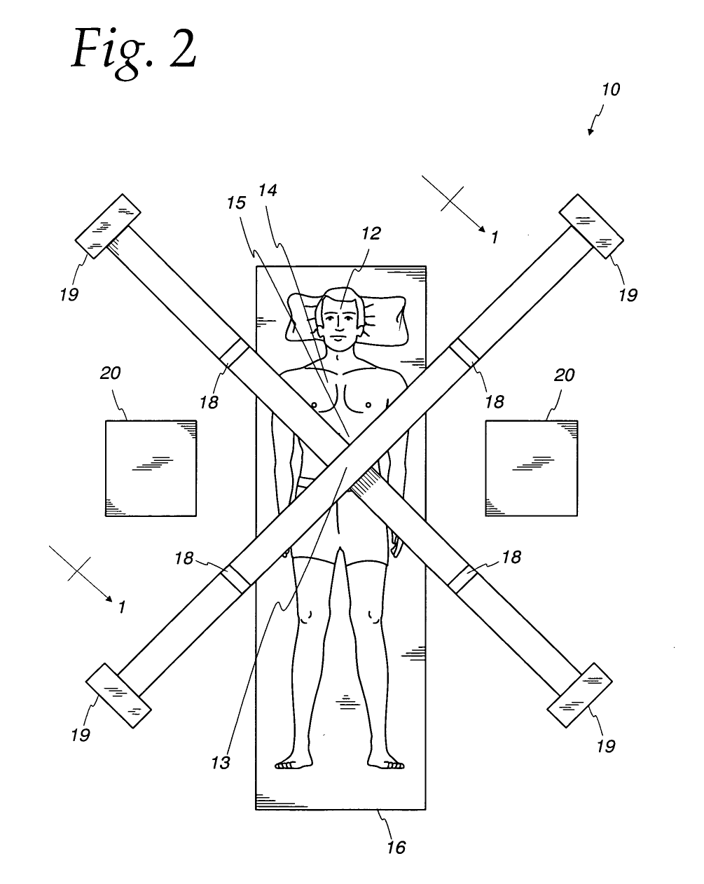

A three-dimensional scan of the source 15 can be accomplished with two lens/detector assemblies 17. FIG. 2 is an exemplary embodiment of a three-dimensional imaging system comprising two intersecting and concentric orthogonal arrays 10 of the lens/detector assemblies. The radiation source 15, resting on a movable platform 16, is located at the intersection of the two arrays at their common center 13 at the time of imaging. Prior to high resolution imaging operations, conventional scintillation counters 20 are provided for quick scan capabilities of the radiating area to approximately locate the source's position. For the sake of additional clarity, FIG. 1 is an elevational view of FIG. 2 taken along lines 1-1.

If the present invention is used as a medical imaging system, then the source 15 is a patient in whom a radioisotope has been injected. A reference source 14 of the same isotope is positioned at a suitable point on the patient's body and the location of the patient's tumor is measured with respect to the reference source 14. Imaging of an extended source is best accomplished by moving the movable platform 16 across the center 13 of the intersecting arrays 10. Alternatively, one could move the lens system relative to the source if means have been provided therefor.

The positions of the lenses, detectors, and a platform 16 containing the source 15 and the reference source 14 are monitored by conventional electronic sensors (not shown) and recorded and analyzed by a computer (not shown).

Lens/Detector Assembly Detail

Each lens/detector assembly 17 incorporates a plurality of movable focussing lenses 18 and detector arrays 19. The positions of the lenses, detectors and a platform 16 containing the source 15 are monitored by conventional electronic sensors (not shown) and recorded and analyzed by computer (not shown).

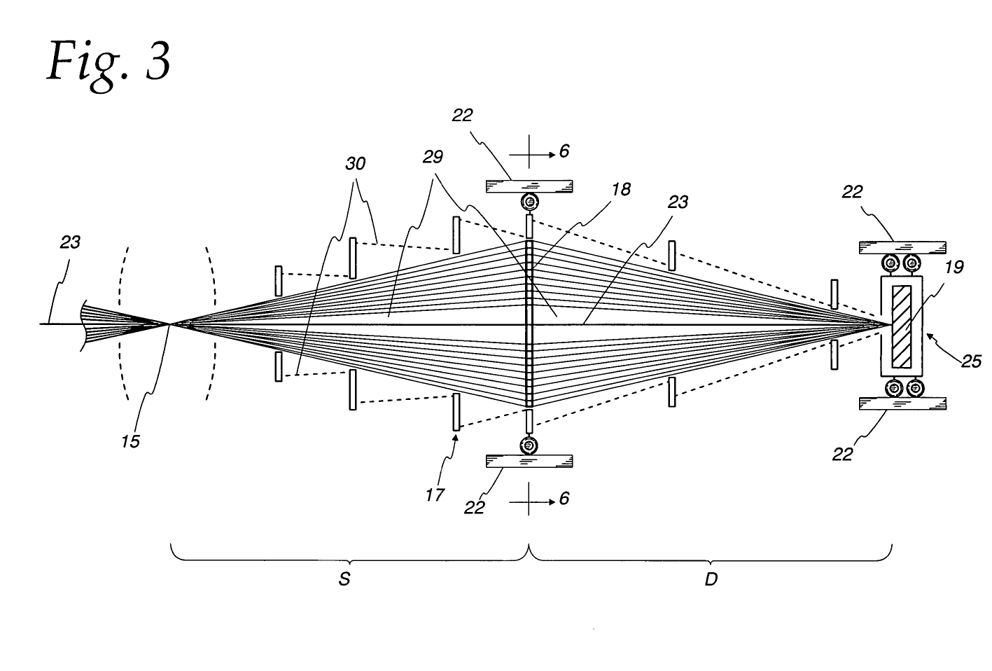

FIG. 3 is a cross sectional view of FIG. 1 taken along lines 3-3 and presents a detailed depiction of the lens/detector assembly 17. Each lens/detector assembly 17 incorporates a plurality of movable focussing means (i.e. the “diffraction lenses” 18), detectors 19, and shielding around the detectors 19. Shielding is also placed along the longitudinal axis 23 of the assembly and longitudinally along the outside of the assembly. The axis and outside radiation shields 29, 30 respectively, are cone-shaped and mounted between the lens 18 and the source 15 and the lens and the detector array 19. Generally, the axis and outside shields can be any convenient configuration such as cone- or cylindrically-shaped. Lead, iron, and brass are suitable shielding materials. S and D denote the lens-source, and the lens-detector distances respectively. Lenses and detectors are mounted on tracks 22 equipped with electronic sensors. The tracks allow for independent axial movement of either or both the lens 18 and detector 19. Typically, the detector array is moved in the same direction but twice as far as the lens.

Detector Detail

Generally, the detector array 19 comprises solid state detectors made of silicon or germanium or a composite material such as CdTe. When radiation is absorbed by these detectors, positive and negative charges are generated that can be collected and measured with suitable electronics. These detectors have much better energy resolution and thus lower background counting rates than scintillation detectors. Thus these detectors allow one to detect weaker sources. These detectors are, however, much more expensive than sodium iodide crystals, zinc sulfide crystals, anthracene, or some other substance (or combination of substances) that scintillates when contacted with ionizing radiation. Also comprising each scintillation detector is a photomultiplier tube to monitor the scintillations as they occur.

A 3 by 3 detector array enables a determination as to whether the source being imaged is on the axis of the lens or off the axis of the lens and if off-axis, to determine in which direction it is off-axis. One may also use a 2 by 2 array, where the source is on axis when the counting rate in all four segments is equal. In the 3 by 3 array, the source is on axis when most of the radiation interacts with the central detector and the other detectors have equally weak count rates. The 3 by 3 array can also be used to obtain the lowest background possible. If the center detector is large enough to intercept all of the focused radiation when the source is on axis, then one needs to consider only the background in the center detector. Furthermore, an energy sum coincidence can be made between the center detector and the outside detectors that can increase the efficiency for detecting the full energy of the gamma ray, thus increasing the full energy count rate without increasing the background count rate. Thus, one has the efficiency of a large detector for detecting the full energy of the gamma ray, while retaining the low background counting rate of only the central detector. An array comprising a large number of elements can also be used (See FIG. 17). In general terms, the size of a an element in a detector array should be equal to or less than the desired spatial resolution.

The Crystal Diffraction Process

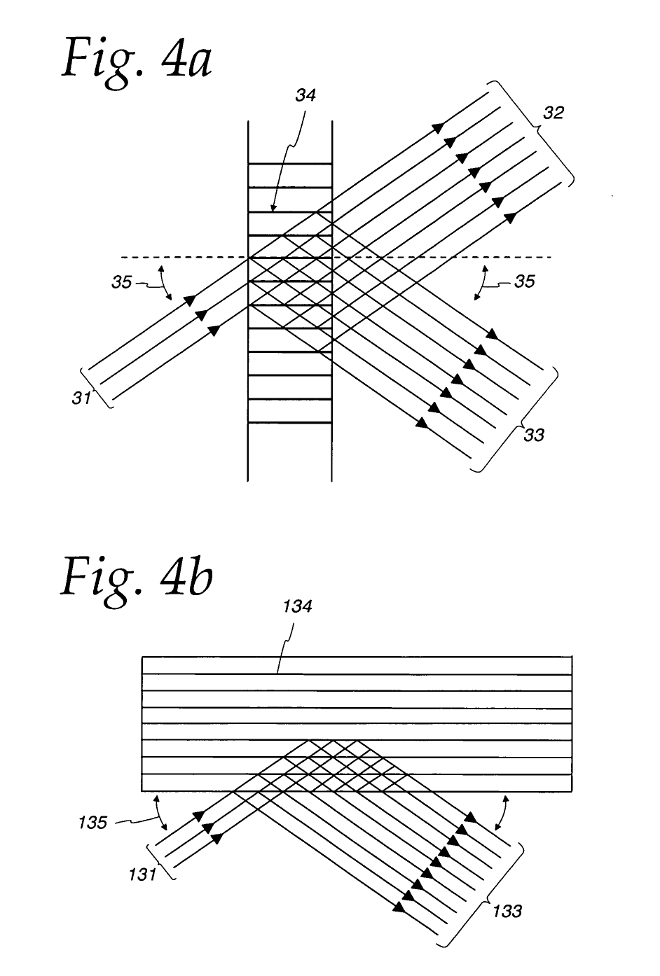

In order to focus x-ray and gamma radiation, the present invention utilizes the phenomenon of crystal diffraction which is illustrated in FIG. 4. FIG. 4a depicts the phenomenon known as Laue diffraction. The incident radiation beam 31 enters through one surface of a diffracting crystal. After interacting with a specific array of parallel atomic layers 34, the radiation beam is split into two beams, a transmitted beam 32, and a diffracted beam 33, with both beams exiting through a surface opposite to the one through which the radiation entered. Both the transmitted and the diffracted beams are produced by a coherent superposition of scatterings by atoms in the parallel crystal layers. The angle 35 between the radiation beam and the crystal layers is designated as p. The maximum fraction diffracted by crystals with some mosaic structure using Laue diffraction is 50%, with the remaining fraction being transmitted without deflection. Typically between 10⁴ and 10⁷ atomic layers are suitable to approach 50% diffraction. The actual number of layers depends on the wavelength of the gamma rays and the width of the mosaic structure of the crystal. In practice, the maximum diffracted beam is less than 50% because some absorption of the beam occurs as it passes through the crystal.

FIG. 4b depicts the phenomenon known as Bragg diffraction acting upon an incident beam 131. After multiple scatterings with the atoms comprising a specific array of parallel atomic layers 134, the net outcome is the emergence of a “diffracted” beam 133, which contains nearly all of the incident energy. Some absorption of the radiation occurs during this process which continues until either the radiation is diffracted out of the crystal or is absorbed in the crystal. The angle 135 between the radiation beam and the crystal layers is designated as p. The diffracted beam exits through the same surface as the one through which the radiation entered. Again, the beam is produced by a coherent superposition of scatterings by atoms in the parallel crystal layers. Bragg diffraction is most effective for energies below 200 keV and the fraction diffracted can reach 90%.

For both Laue and Bragg diffraction, diffraction occurs only when the Bragg condition is obeyed, (equation 1):
λ=2d_{hld} sin p (1)
where λ is the radiation wavelength, d_{hld} the spacing between the atomic layers indicated by the Miller indices h,k,l, and p the angle between the direction of the radiation beam and the atomic layers (one can convert energy E in keV to wavelength λ in Angstrom units by using the relation λ=12.397/E). With perfectly parallel atomic layers, only rays within a few arc seconds of p will be diffracted (i.e., the “acceptance angle” is only a few seconds of arc), so that one can obtain a large diffraction efficiency only if the rays are nearly parallel, i.e. only if the source is very far away.

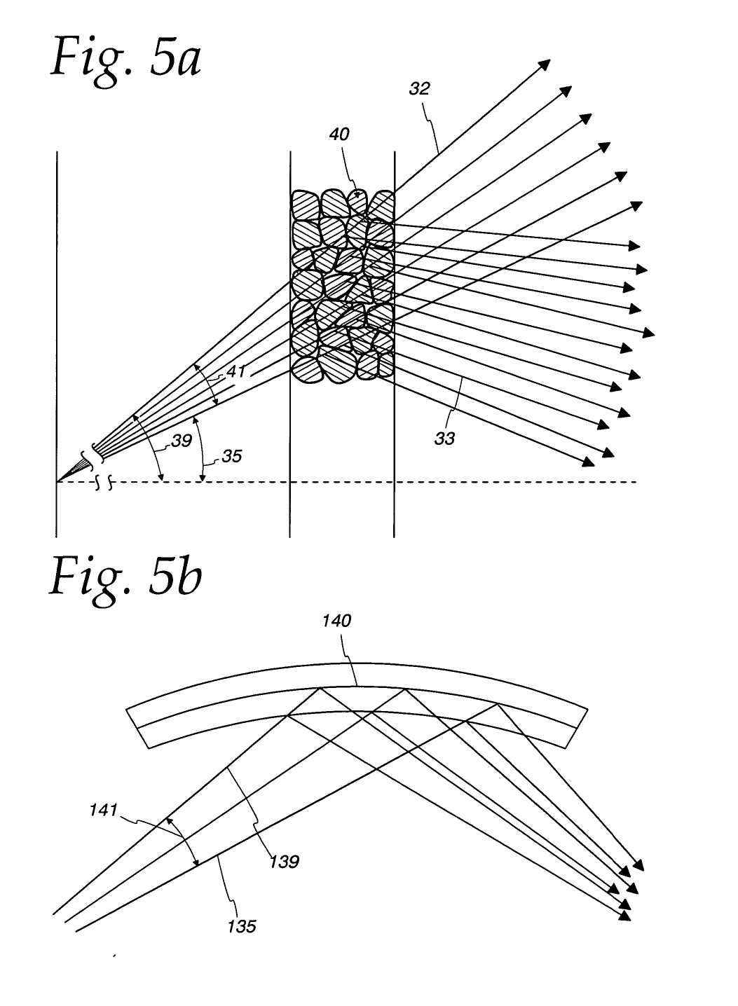

As seen in FIG. 5a, this problem can be overcome (i.e. the acceptance angle can be increased). FIG. 5a shows that for Laue diffraction, if imperfections are either naturally present or else artificially introduced within the crystal so that all the crystal planes are no longer parallel to each other, rays coming at different angles 39 will still find planes 40 for which the Bragg condition is obeyed. As seen in FIG. 5a the imperfections in the crystal give rise to a three dimensional mosaic structure. The angle 41 between the rays 35 with the lowest angle p and those 39 with the largest p is the acceptance angle (also known as the “rocking angle”). Ordinarily, rocking angles of between 200 and 800 arc seconds are employed. This is adequate for a first scan where a spatial resolution of 4 mm suffices. A rocking angle of between 50 and 150 seconds of arc is required when a 1 mm spatial resolution is required. The use of crystals with a smaller rocking angle is not indicated unless other components of the system are adjusted so as to yield a better than 1 mm resolution (e.g. reduction of the sizes of the detectors and the source and detector apertures).

FIG. 5b shows that for Bragg diffraction the acceptance angle can be increased if the crystal is curved in the direction of the radiation beam. Rays coming at different angles 139 will still find planes 140 for which the Bragg condition will be obeyed. The angle 141 between the rays 135 with the lowest angle p and the rays 139 with the largest p is the acceptance angle. The curved shape of the crystals produces a significant focusing effect. The highest degree of focusing for Bragg diffraction occurs when the radius of curvature is equal to L/sin p, where L is the distance from the source to the lens. Furthermore, a mosaic structure in the crystal produces an increase in the acceptance angle in the same manner as described above for Laue diffraction.

The Diffraction Lens Construction and Use

Each crystal diffraction lens 18 utilizes a plurality of diffracting crystals. Possible crystalline materials include, but are not limited to, silicon, quartz, tin, molybdenum, germanium, and copper.

FIG. 6 is a view of FIG. 3 along lines 6-6 depicting a typical embodiment of a lens 18 in the Laue diffraction configuration. Each lens 18 comprises a support substrate 43 typically a metal plate. Stainless steel, brass, tungsten, and aluminum are suitable materials for the substrate 43 with stainless steel, brass, and tungsten having the advantage of better shielding the detector from radiation that was not diffracted by the crystals 42. Regions of the surface of the plate 43 define a series of apertures 44 arranged as concentric rings 45. Each ring contains a plurality of diffracting crystals 42 of the same material and orientation. The material and orientation are determined according to the procedure described below. The innermost ring has a diameter of about 2.7 cm and the outermost ring has a diameter of about 11.6 cm.

FIG. 7 is a view of FIG. 3 along lines 6-6 and depicts a typical embodiment for lens 18 in the Bragg configuration. The significant difference is that the curved apertures 125 are much wider than the corresponding apertures 44 in FIG. 6a.

For both Laue and Bragg diffraction, the diffracting crystals are mounted onto the plate in such a manner that once mounted, all the crystals in a ring will be so oriented as to use the same set atomic layers to satisfy the Bragg condition. In a typical embodiment, the crystals in a given ring are all of the same material but crystals in different rings may be of different materials.

The first step in determining the material and orientation of the diffracting crystals is to select the energy of the radiation that will be observed and the focal length F of the focussing means 18 one wants to achieve. In the simplest embodiment of the invention, a single lens is utilized, in a lens/detector array 17, but a lens/detector assembly 17 having a plurality of lenses is also suitable.

Where lenses of focal length F1, F2, F3, etc . . . are placed in close proximity or contact with each-other, the focal length of the combination is given by equations 2 through 6.

Equation 2 gives the focal length for one lens, where p is the Bragg angle used in the lens and R is the radius of the crystal ring.
F=R/(tan 2 p) (2)

Equation 3 gives the focal length for two lenses, where p₁ and P₂ are the Bragg angles used in the first and second lenses and R1 and R2 are the radii used in the first and second lens, respectively.
F12=(R1−R2)/tan 2 p₁+R2/tan(2 p₁+2 p₂) (3)

Equation 4 gives the focal length for three lenses, where p₁, p₂ and p₃ are the Bragg angles used in the first and second and third lenses and R1, R2 and R3 are the radii used in the first, second and third lenses, respectively.
F123=(R1−R2)/tan 2p₁+(R2−R3)/tan 2(p₁+p₂)+R3/tan 2(p₁+p₂+p₃) (4)

If the lenses are very close together, then the R's become approximately equal and the approximate formula for the focal length is given by equation 5.
F12 . . . n=R(Ave)/tan 2(p₁+p₂+p₃ . . . pₙ) (5)

If all of the Bragg angles are quite small, the focal length can be approximated by equation 6
1/(F12 . . . n)=1/F1+1/F2+ . . . +1/Fn (6)

The set of atomic layers to be used for each ring 45 is determined by the condition that all the rings must have the same focal length F. For rays near the lens axis (small p) the relation between lens-source distance S, lens-detector distance D, and focal length F is given approximately by equation 7.
(1/F)=(1/S)+(1/D) (7)

In practice S and D as shown in FIG. 3 are both chosen to be 2F and the image formed onto the detector array is about the same size as the source if the source is larger than the crystal elements in the crystal. If the crystals are bigger than the source, the image will be about 1.6 times the size of the crystals. Then the Bragg angle p is arc tan[R/(2F)] where R is the radius of the ring. The Bragg condition yields the relation between the ring radius, focal length, radiation wavelength λ and atomic layer spacing d, given by equation (8).
R/F=tan[2 arc sin(λ/2d_{hld})] (8)

For F>>R, i.e., for small angles, Equation 8 yields
d_{hld}=λF/R (9)

In practice, a gamma ray with a specific energy (and therefore wavelength λ) is selected. Then, the crystalline plane spacings of an available crystal are tabulated. This information is combined with the desired focal length F to arrive at the respective radii R for the crystal rings, pursuant to equation 10:
R=d_{hld}/λF (10)

Finally, the size of the crystals is chosen.

Alternately, λ is determined from the desired gamma ray energy, then F is chosen, and the available values of d_{hld} are identified, so that the values of R for the rings are suitable. Copper and germanium are suitable for radiation energies above 100 keV. Lower atomic number materials such as quartz, silicon, and beryllium are more suitable for low energy gamma rays (below 100 keV.)

Laue Diffraction Lens Detail

In a preferred embodiment for a Laue diffraction lens, copper crystals grown at and obtainable from a facility such as the Institut Langevin-Langmuir (ILL) in Grenoble, France, are utilized. Copper crystals naturally exhibit enough imperfections in their crystal lattice so that their acceptance angle (“rocking angle”) is of the order 200 to 500 seconds of arc, i.e. between 0.06 and 0.15 degrees. Heating and then compressing copper crystals increases the acceptance angle even further. On the other hand one may obtain crystals with rocking angles with acceptance angles of 50 arcseconds or less.

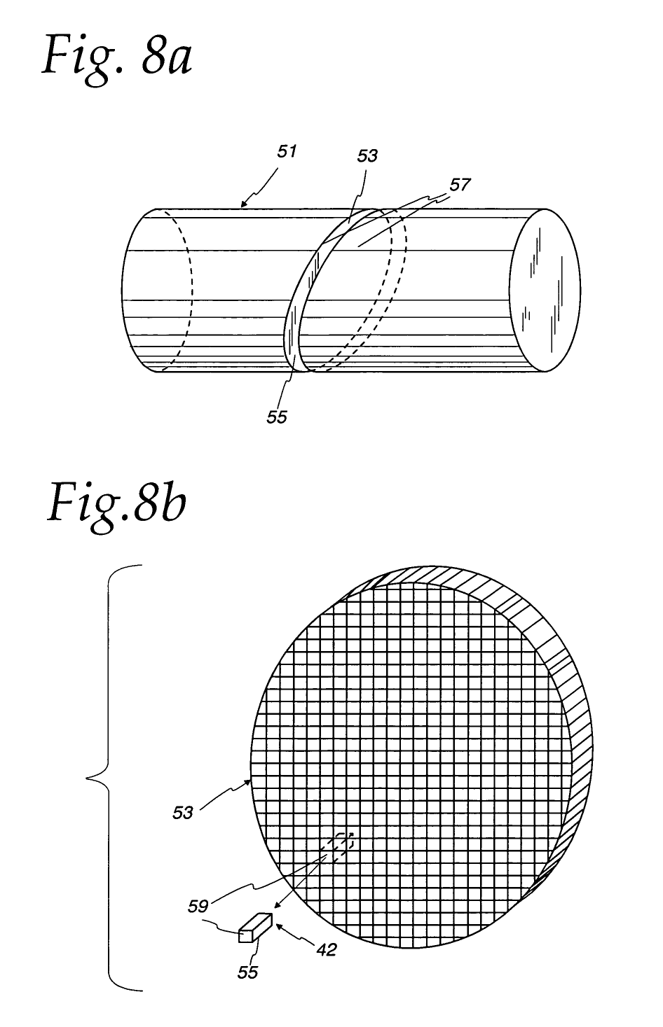

Referring to FIG. 8a, ILL typically provides cylindrical copper crystals 51 of 10 cm. in diameter and 25 cm. long, with a predetermined crystal orientation. Thin slabs 53, of 1 mm thickness 55 or less, are cut parallel to the planes 57 designated by the Miller indices that have been selected. As shown in FIG. 8b, the slabs 53 are then cut in turn into crystals 42 with faces 59 approximately 1 mm or less wide. The faces 59 are perpendicular to the planes 57. If the source is small, it is critical that the faces 59 be at least as small. Experiments have shown that If the crystals are bigger than the source, the image is about 1.6 times the size of the crystals. In the '841 patent embodiment 4 mm square faces where used yielding a 7 mm image of the source.

Mounting of the crystals 42 onto the plate 43 can occur in a variety of ways. One way is to first place the plate 43 against a rigid flat surface so that the flat surface is accessible though the concentric ring apertures 45. A number of crystals 42 are placed on the flat surfaces within the confines of the ring 45 with the face 59 of the crystals 42 (i.e., the face that corresponds to the plane selected as described supra and through which the radiation enters the crystal) flush against the rigid flat surface. Enough crystals are placed in the ring to virtually fill the ring aperture. The crystals are then cemented together. Upon completion of the mounting procedure, the face 59 of the crystals that is perpendicular to the planes 57 whose Miller indices have been selected is perpendicular to the lens/detector assembly axis 23. (see FIG. 3) The width of this crystal face (1 mm or less wide is suitable, as suggested supra) and the rocking angle of the mosaic structure determine the ultimate size of the image spot at the detector location 19. If the angular width of the elements of the mosaic structure as seen from the source is small compared to the angular width of the crystals, then the spatial resolution of the system is 1.6 times the angular width of the crystals. Copper and Germanium crystals are suitable for radiation energies above 100 keV. Lower atomic number materials such as quartz, silicon, and beryllium are more suitable for low energy gamma rays, i.e., below 100 keV.

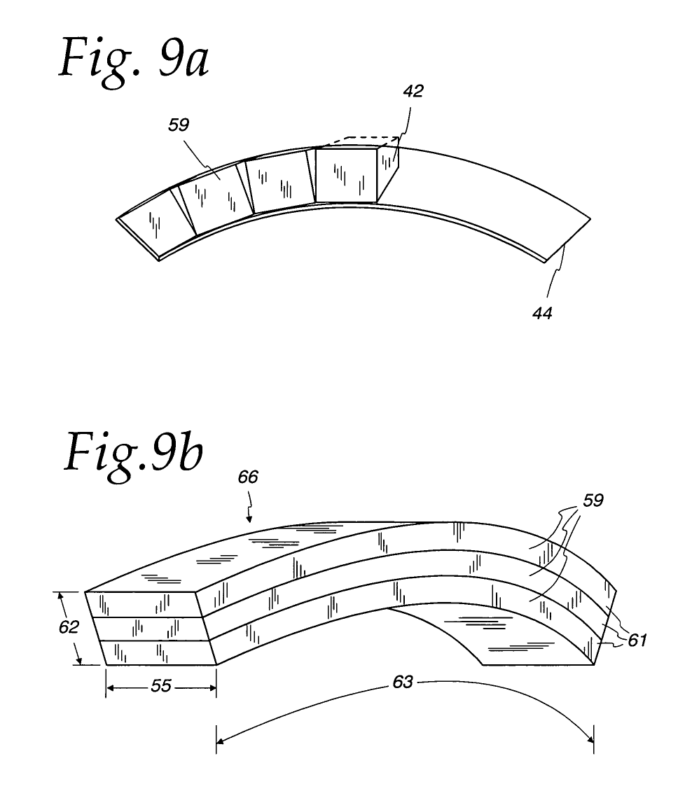

FIG. 9a is a detailed view of one of the curved apertures 44 shown in FIG. 6 that contain the crystals 42. As shown, the crystals can be large enough to entirely fill the face of the aperture 44 or, if smaller than the aperture, they can be stacked on top of each other. In either configuration, once in place the crystals are then cemented into the containing means 44. In the instant embodiment, the containing means is the curved apertures. The face 59 of the crystals that is perpendicular to the planes 57 whose Miller indices have been selected is parallel to the plane of the substrate 43.

Alternatively, as shown in FIG. 9b, one may cut the crystals into thin strips 61 having a length 63 of perhaps 1 to 20 cm. The strips 61 are arranged in stacks 66 of a predetermined height 62 (1 mm or less), and then bent into circular arc sections of the same radius as the ring 45 to be mounted therein. This procedure is more suitable when the crystalline materials are malleable. This procedure has two distinct advantages: cementing the several centimeters long strips is much easier than cementing the 1 mm square crystals and there is much less likelihood that the component crystals will be misaligned and that the radiation they diffract will be directed away from the focal point. Crystal strips may be bent using, inter alia, cold bending, hot bending, bending with a slotted back, or annealing.

Generally, crystals of malleable materials (e.g. copper, molybdenum and tin) or crystals of materials with low melting points exhibit a high degree of mosaic structure. With suitable treatment, however, many other crystal types (and not just those from malleable elements) can be made to exhibit mosaic structure resulting in acceptance angles of 50 to 150 seconds of arc. A crystal with an acceptance angle of 150 arc seconds or less is considered a high quality crystal. Methods for introducing such mosaic structure include neutron irradiation, heating the crystal to near its melting point and then subjecting it to stresses or compression, subjecting the crystal to mechanical vibrations (e.g. sonic vibrations), and introducing impurities (i.e. dopants). Generally, the higher the atomic number of the material, the more efficient it is for diffraction of high energy gamma-rays.

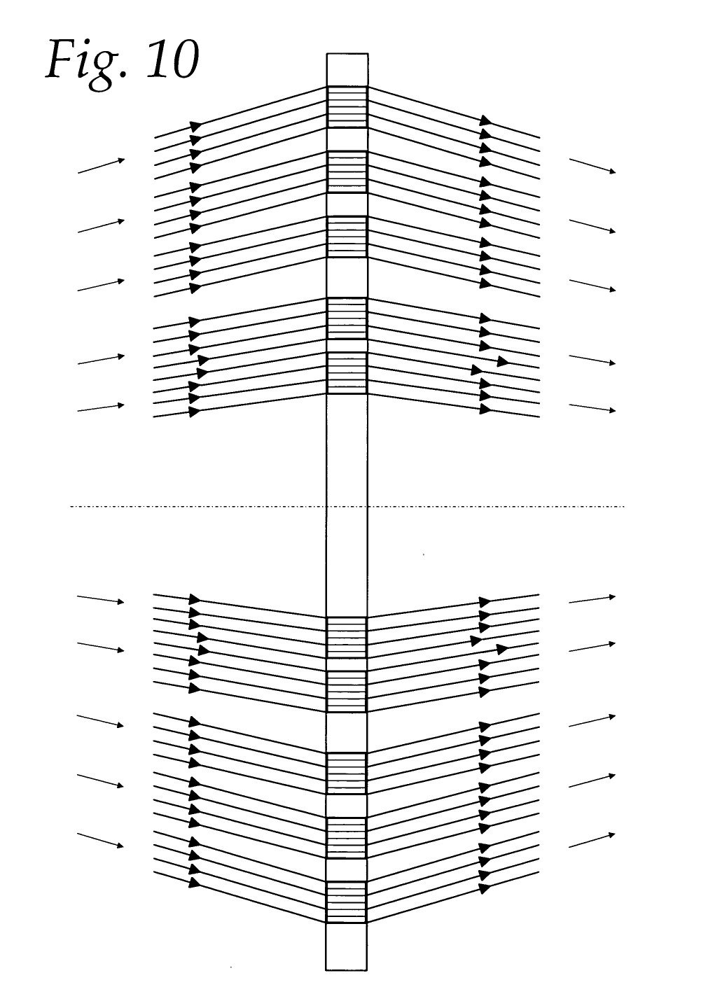

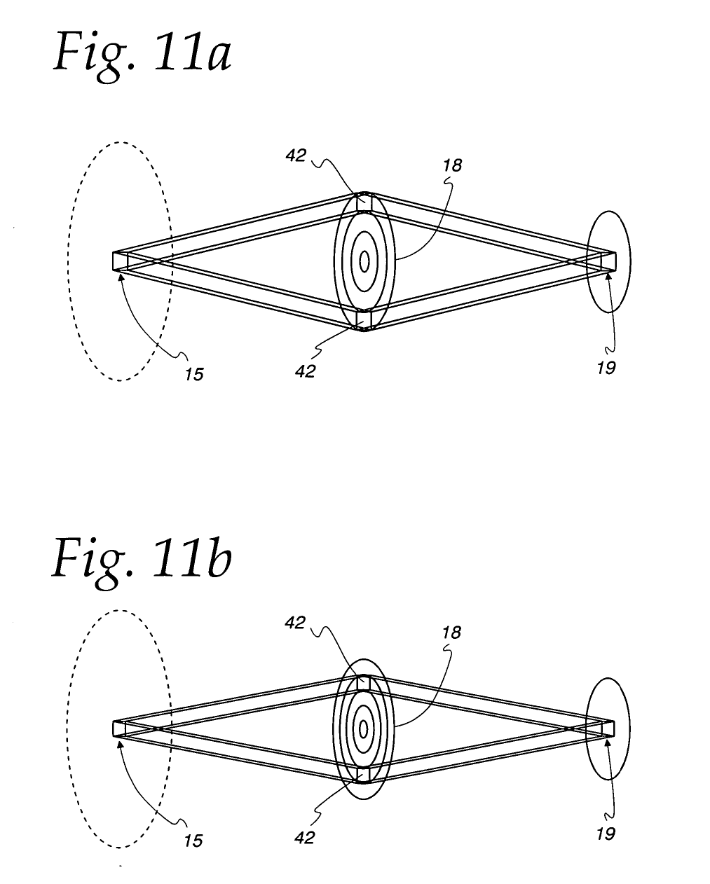

FIG. 10 is a cross-sectional view of a Laue diffraction lens 18 taken along lines 10-10 in FIG. 6 and FIG. 11 illustrates the reduction of field of view at the detector when large lens crystals are replaced by small crystals.

Bragg Diffraction Lens Detail

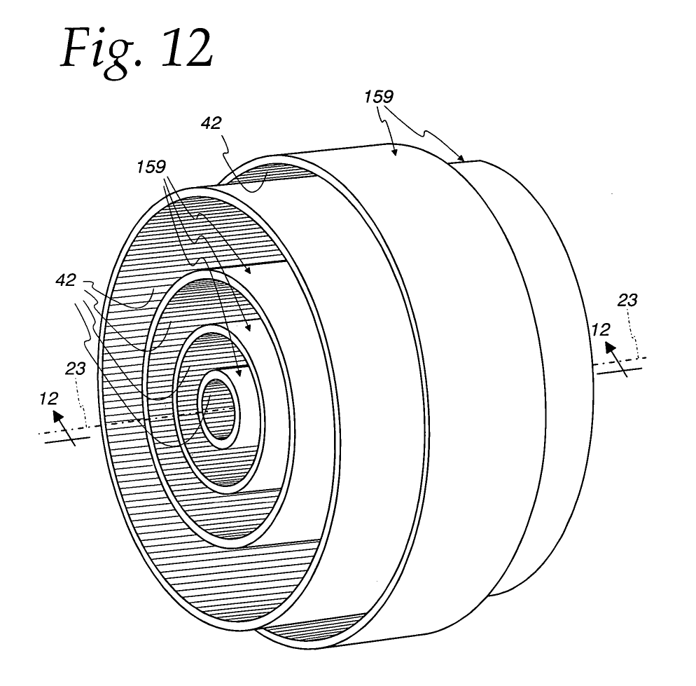

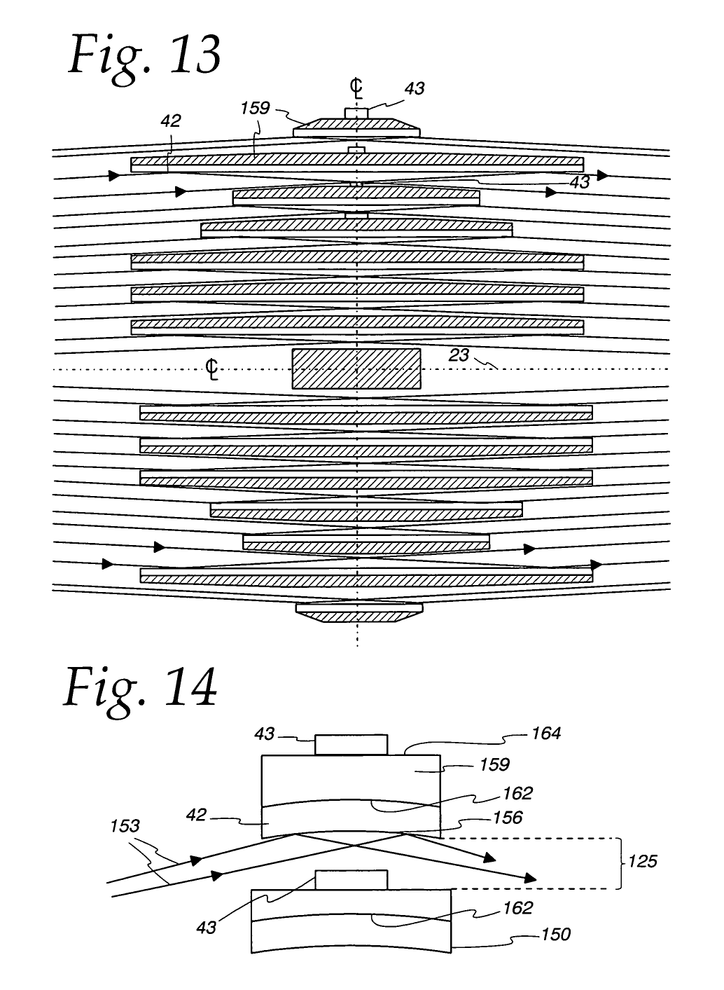

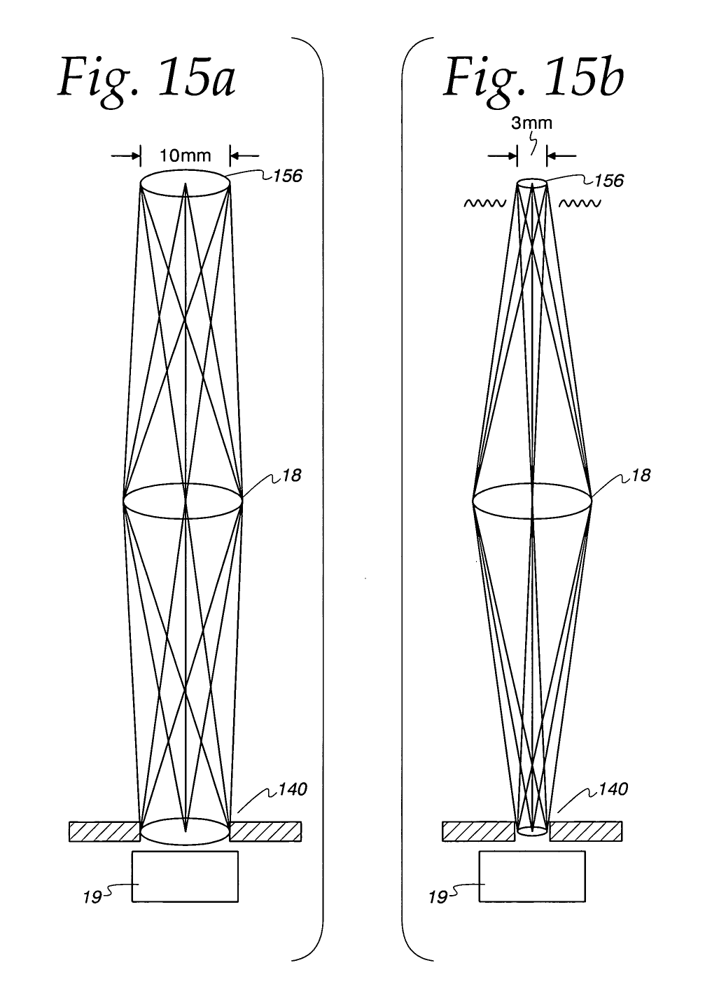

A wide variety of crystals are suitable for a Bragg diffraction. This is because the diffracted beam does not have to pass completely through the crystal and be reduced in intensity by absorption of the full thickness of the crystal. The diffraction efficiency for Bragg diffraction is determined by the ratio of the diffraction coefficient per unit length to the absorption coefficient per unit length. Since the ratio of these two quantities remains nearly the same for low gamma ray energies, the diffraction efficiency does not change as dramatically with changing energy as it does in the Laue diffraction case. For Bragg diffraction with bent crystals 42 (see FIG. 12 and FIG. 13), a large mosaic structure is not required in order to achieve a large acceptance angle. All that is required is that one be able to cut the crystals to form bendable strips of suitable dimensions. Exemplary dimensions are strips 1 mm wide, 0.5 to 1 mm thick, and 2 to 20 cm long. The crystal strips are then bent to a radius of 1 m or more, perpendicular to the crystal's long axis. The needed radius of curvature is equal to the distance from the source to the lens divided by sin p. The typical length of a strip is given by the width of the aperture 125 divided by sin p.

In the case of the bent Bragg crystals, the width of the mosaic structure controls the size of the field of view of the lens. Thus, one can adjust the size of the field of view independently of the size of the solid angle subtended by the crystals. Crystalline planes are selected and crystal strips are cut in much the same way as described in conjunction with Laue diffraction.

FIG. 11 is a three dimensional view of a Bragg lens. The Bragg crystals 42 are mounted on the concave surfaces of a plurality of coaxial cylindrical supports 159. FIG. 12 is a cross-sectional view of FIG. 11 along lines 12-12, and FIG. 13 is a detailed view of FIG. 12. FIG. 13 depicts how the crystals 42 are mounted on the supports 159. Said supports 159 are in turn mounted in a substrate 43 containing apertures 125 arranged as concentric rings 45. As can be seen in FIG. 13, the apertures 125 corresponding to each ring 45 are much wider than the crystal thickness 150 in order to allow the radiation beam 153 to impact upon all of the crystal face 156. The supports 159 that are provided for the bent crystals are shaped so that the radius of the support surface 162 matches that of the bent crystal. One such support can be a machined surface integrally molded, or removably attached to the substrate 43.

The use of bent Bragg crystals in the lens allows one to focus the diffracted beam from an individual crystal into a narrow line parallel to the diffraction planes and on the axis 23 of the assembly 17. This concentrates the diffracted beams from the full lens and makes it possible to use smaller detectors in the focal plane. The length 162 of the crystals 42 and the supports 159 is 2 to 20 cm in the direction of the beam 153. Generally, the longer crystals are closest to the lens axis 23, where the values of sin p are smallest. The length of the crystal strips are adjusted to obtain the maximum diffracted flux.

Scanning a Source and Formation of an Image

The lens detector assembly achieves its best performance for sources located on or very near the axis of the assembly. When the source 15 is not situated on the axis of the assembly, the movable platform 16 is advanced until the source is positioned on the axis of the assembly.

In order to scan across the source 15, one may change the position of the body 12 using the means provided for moving the table 16. Alternatively, one may change the orientation of the lens/detector assemblies and adjust the source/lens and lens/detector distances as indicated by Equation 3 by means of the tracks 22 on which lenses and detectors are mounted. In yet another alternative, one can move the whole lens system relative to the source.

Also, equation 5 shows the focal length's dependence on the wavelength of the radiation. The lens 18 and detector 19 are mounted on tracks 22 allowing the use of a given lens to detect radiation of a different wavelength by adjusting lens-source and lens-detector distances as dictated by equation 3. Electronic sensors are mounted on tracks 22 and their signals are recorded and analyzed by the computer.

Instead of relying on tracks 22, imaging of radiation of different wavelengths can also be accomplished by using different lenses, and keeping the elements of the assembly stationary. For example, a source having a first energy can be scanned in toto by moving the table 16 with respect to the center of the lens/detector arrays (see FIG. 1). If the device is to be used for gamma rays of a second energy, one can construct a plurality of different lenses using crystals with atomic spacings so chosen that one obtains the same focal length as the lenses used to focus the first source.

Signals from the detector arrays 19 are analyzed by a computer in conjunction with the data from the detectors 20 and those from the sensors on the movable platform 16 and the lens and detector tracks 22.

Appropriate Diffraction Materials

A variety of crystalline materials (Germanium, Silicon, Copper and Quartz) have been found by the applicant to be suitable for the fabrication of x-ray and gamma-ray lenses for energies of around 150 keV.

Device Apertures

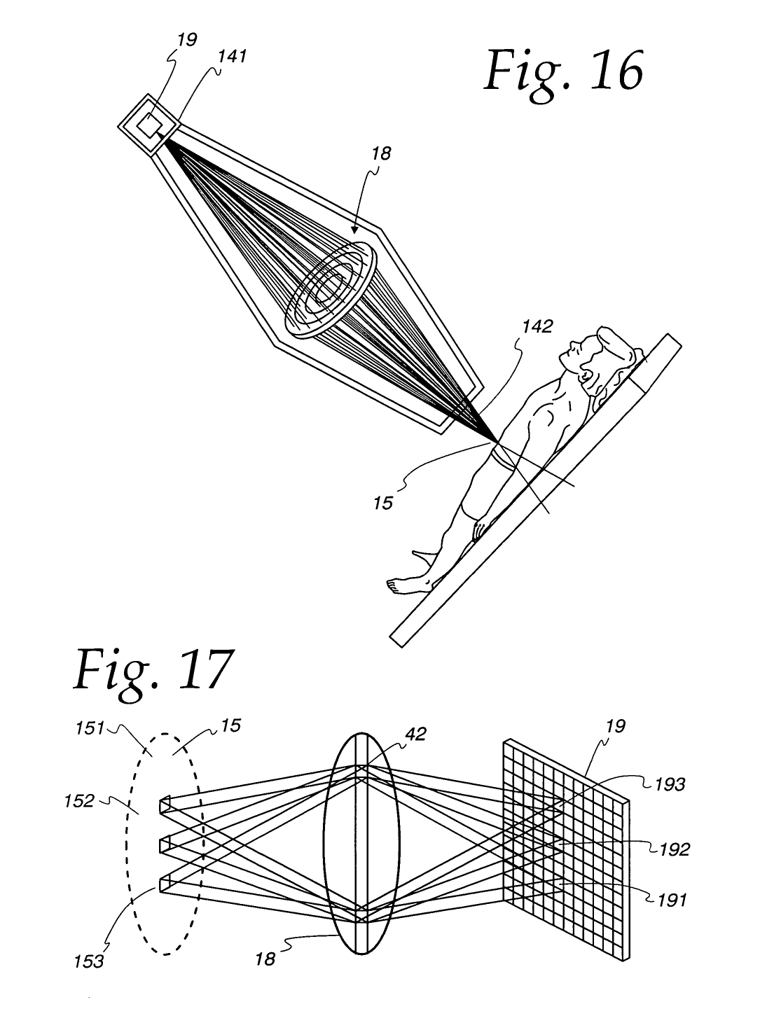

Restricting the area of the apertures in front of the detector array and in front of the source improves significantly the spatial resolution of the device. As shown in FIGS. 14a and 14b, a decrease in the detector array aperture 140 reduces the field of view, i.e. the area 156 of the source 15 that can be viewed at any one time. Where the angular width of the mosaic structure is small compared to the angular width of the detector aperture, the smaller the detector aperture, the smaller the apparent size of the source. In a series of observations, the inventor has shown that a 3 mm detector aperture produces a 3 mm FWHM image of a 1 mm source. This restriction of the detector aperture also reduces the background seen by the detector array. Restriction of the source aperture 142 has a similar advantage in reducing the apparent size of the source and in reducing the background radiation reaching the detector array. FIG. 16 illustrates the combined effect of narrow apertures in front of the detector array and in front of the source.

It is advisable that the size and position of the apertures be adjustable so that one may adapt the device to the specific requirements for a given observation.

Use of a Multi-Component Detector Array

FIG. 17 depicts an alternate embodiment of the invention where using a many-component detector array presents the same spatial resolution advantages as the restriction of the detector and source apertures. FIG. 17 shows how points 151, 152, and 153 are imaged onto separate detectors 191, 192, 193. Such an array has obvious advantages in that it reduces the time necessary to acquire the necessary data. For instance, a 1 cm square array of 1 mm by 1 mm detectors produces a high spatial resolution (1 mm) life size image of a 1 cm object such as a tumor without having to scan the object.

While the invention has been described with reference to details of the illustrated embodiment, these details are not intended to limit the scope of the invention as defined in the appended claims.

1. A method for high spatial resolution imaging of x-ray and gamma radiation comprising:
a) supplying a plurality of sources of radiation; and
b) focussing said radiation onto one or more detectors by means of diffracting crystals having a width not exceeding the resolution;
c) analyzing said focused radiation to collect data as to the type and location of the radiation; and
d) producing an image using the data.

2. The method as recited in claim 1 wherein the step of supplying said sources of radiation further comprises contacting a body with a radioisotope.

3. The method as recited in claim 1 wherein said image is produced by an array of detectors.

4. The method as recited in claim 1 wherein the step of arranging said crystals further comprises cutting said crystals into thin slabs and bending said cut crystals to assume the shape of circular arcs.

5. The method as recited in claim 1 wherein said width is 1 mm or less.

6. The method as recited in claim 1, further comprising selecting said crystals to have random imperfections and dislocations produce a rocking angle of between 50 and 150 seconds of arc.

7. The method as recited in claim 1 wherein the step of analyzing said focused radiation further comprises directing said focused radiation to a plurality of detectors with a resolution of 1 mm or less.

8. The method as recited in claim 1 further comprising positioning one or more collimators with narrow width apertures between said sources and said detectors and adjusting the position and widths of said apertures to improve said resolution.

9. The method as recited in claim 4 wherein the step of supplying a plurality of said sources of radiation further comprises placing at least one of said sources at precisely known locations.

10. A device for high spatial resolution imaging of a plurality of sources of x-ray and gamma-ray radiation comprising:
a) a means for locating the sources of radiation;
b) a plurality of diffracting crystals of a width not exceeding the resolution for focussing the radiation emanating from the located sources and directing it to a plurality of detectors;
c) detector arrays for analyzing said directed radiation to collect data as to the type and location of the radiation; and
d) a means for converting the data to an image.

11. The device as recited in claim 10 wherein the means for locating the sources is a plurality of scintillation devices.

12. The device as recited in claim 10 wherein the diffracting crystals form a plurality of lenses.

13. The device as recited in claim 10, where the diffracting crystals and the sources are movable.

14. The device as recited in claim 10 wherein the radiation sources are radioisotopes.

15. The device as recited in claim 12 wherein each lens comprises a plurality of crystals and wherein the crystals are oriented so as to diffract radiation of a predetermined energy to the same focal point.

16. The device as recited in claim 12 wherein the crystals are mounted in concentric rings onto a substrate.

17. The device as recited in claim 12 wherein the crystals are bent.

18. The device as recited in claim 10 further comprising one or more collimators with apertures positioned medially between the sources and the detectors and wherein the width and position of said apertures is adjustable.

19. The device as recited in claim 10 wherein said crystals contain random imperfections and dislocations that produce a rocking angle of between 50 and 150 seconds of arc.

20. The device as recited in claim 10 wherein said detectors in said detector arrays have a resolution of 1 mm or less.
